(19) United States
(12) Patent Application Publication
(10) Pub. No.: US 2014/0120046 A1
(43) Pub. Date: May 1, 2014

(54) METHODS FOR MAKING CLEAR STICK ANTIPERSPIRANT COMPOSITIONS AND PRODUCTS

(71) Applicant: THE DIAL CORPORATION, Scottsdale, AZ (US)

(72) Inventors: Berea Thompson, Somerset, NJ (US); Travis T. Yarlagadda, Phoenix, AZ (US); Catherine Schmit, Glendale, AZ (US); Debra K. Butterworth, Scottsdale, AZ (US)

(73) Assignee: The Dial Corporation, Scottsdale, AZ (US)

(21) Appl. No.: 13/666,630

(22) Filed: Nov. 1, 2012

(51) Int. Cl. A61K 8/34; A61Q 15/00; A61K 8/58

(52) U.S. Cl. 424/66; 424/65; 424/68

(57) ABSTRACT

Methods for making clear stick antiperspirant compositions and products are provided herein. In one example, a method for making a clear stick antiperspirant composition comprises the steps of providing a solvent carrier at substantially below room temperature to define a cool solvent carrier. A gelling agent comprising dibenzylidene sorbitol is incorporated into the cool solvent carrier to form a solvent/DBS premix. An active antiperspirant compound and the solvent/DBS premix are combined.

### TECHNICAL FIELD

The present invention relates generally to antiperspirant compositions and products, and more particularly relates to methods for making clear stick antiperspirant compositions and products.

### BACKGROUND

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, etc.

Stick antiperspirant products include an antiperspirant composition within a container. During use of the stick antiperspirant product, the top of the container is removed and the application surface of the composition is contacted with the skin, such as the underarm, by swiping or rubbing the stick across the skin. Sometimes the product also includes an undercap, or factory seal, that covers the application surface and is removed prior to first use. The container generally also includes some mechanism for moving the composition upwards through the container to continue to provide an exposed application surface.

Clear stick antiperspirant products are a type of antiperspirant product that includes a clear stick. A “clear stick” as used herein, is understood to mean a stick that is visually clear so that, like glass, it allows ready viewing of objects behind it. The compositions for these products typically include, among other ingredients, a liquid solvent vehicle or carrier, an antiperspirant salt, and a gelling agent. Dibenzylidene sorbitol is a gelling agent that can be used for such stick products because it effectively gels or solidifies the solvent carrier to form a visually clear stick. Unfortunately, it can be difficult to fully incorporate dibenzylidene sorbitol into the solvent carrier during manufacturing, thereby making the production of clear stick antiperspirant products more challenging.

Accordingly, it is desirable to provide methods for making clear stick antiperspirant compositions and products that facilitate fully incorporating a gelling agent comprising dibenzylidene sorbitol into a solvent carrier. Furthermore, other desirable features and characteristics of the present invention will become apparent from the subsequent detailed description and the appended claims, taken in conjunction with the accompanying drawings and this background.

### BRIEF SUMMARY

Methods for making clear stick antiperspirant compositions and products are provided herein. In an exemplary embodiment, a method for making a clear stick antiperspirant composition is provided. The method comprises the steps of providing a solvent carrier at substantially below room temperature to define a cool solvent carrier. A gelling agent comprising dibenzylidene sorbitol is incorporated into the cool solvent carrier to form a solvent/DBS premix. An active antiperspirant compound and the solvent/DBS premix are combined.

In accordance with another exemplary embodiment, a method for making a clear stick antiperspirant composition is provided. The method comprises the steps of mixing a cool amount of a solvent carrier and a gelling agent that comprises dibenzylidene sorbitol at a first temperature that is substantially below room temperature to form a solvent/DBS premix. An active antiperspirant compound and the solvent/DBS premix are mixed at a second temperature to form a solvent/gellant-antiperspirant mixture. The second temperature is greater than the first temperature. The solvent/gellant-antiperspirant mixture is heated to a third temperature that is greater than the second temperature to form the clear stick antiperspirant composition in molten form.

In accordance with another exemplary embodiment, a method for making an antiperspirant product is provided. The method comprises the steps of forming a clear stick antiperspirant composition. A gelling agent that comprises dibenzylidene sorbitol is incorporated into a cool solvent carrier that is substantially below room temperature to form a solvent/DBS premix. An active antiperspirant compound and the solvent/DBS premix are combined to form a solvent/gellant-antiperspirant mixture. The solvent/gellant-antiperspirant mixture is heated to form the clear stick antiperspirant composition in molten form. The clear stick antiperspirant composition is deposited into a mold. The clear stick antiperspirant composition is allowed to solidify.

### BRIEF DESCRIPTION OF THE DRAWINGS

The various embodiments will hereinafter be described in conjunction with the following drawing figures, wherein like numerals denote like elements, and wherein:

FIG. 1 is a perspective view of a clear stick antiperspirant product in accordance with an exemplary embodiment;

FIG. 2 is a top view of the clear stick antiperspirant product depicted in FIG. 1;

FIG. 3 is an exploded perspective view of a clear stick antiperspirant product in accordance with an exemplary embodiment; and

FIG. 4 is a flowchart of a method for making a clear stick antiperspirant product in accordance with an exemplary embodiment.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

The various embodiments contemplated herein relate to methods for making clear stick antiperspirant compositions and products. Unlike the prior art, the exemplary embodiments taught herein form a clear stick antiperspirant composition by providing a solvent carrier at substantially below room temperature to define a cool solvent carrier. As used herein, the phrase “room temperature” refers to temperatures from about 20 to about 25° C., and the phrase “substantially below room temperature” refers to temperatures at least several degrees below 20° C. In one embodiment, the solvent carrier is provided and/or cooled to a temperature of about 15° C. or less to define the cool solvent carrier. A gelling agent comprising dibenzylidene sorbitol (DBS) is incorporated, e.g., via mixing, into the cool solvent carrier to form a solvent/DBS premix. It has been surprisingly found that dibenzylidene sorbitol more readily disperses and/or suspends in certain solvent carriers, such as polyhydric alcohols (e.g., propylene glycol and the like), when the solvent carrier is cooled to a temperatures substantially below room temperature in contrast to more conventional approaches in which the gelling agent is dispersed and/or suspended in a room temperature or heated solvent carrier. As such, the solvent/DBS premix comprises dibenzylidene sorbitol substantially incorporated (e.g., dispersed and/or suspended) in the cool solvent carrier. Also surprisingly found was that maintaining the solvent/DBS premix at cooler temperatures helps maintain the premix at a relatively low viscosity compared to the viscosity of the premix at higher temperatures, thereby facilitating pumping or transferring of the premix as needed for combining with other antiperspirant ingredients to form a main batch of the clear stick antiperspirant composition. An active antiperspirant compound and optionally other antiperspirant ingredients are subsequently combined with the solvent/DBS premix to form a solvent/gellant-antiperspirant mixture. In an exemplary embodiment, the solvent/gellant-antiperspirant mixture is heated to substantially solubilize the ingredients including the dibenzylidene sorbitol into the solvent carrier to form the clear stick antiperspirant composition in a molten form. In an exemplary embodiment, the clear stick antiperspirant composition is deposited into a container, for example, and allowed to solidify to form a clear stick antiperspirant product.

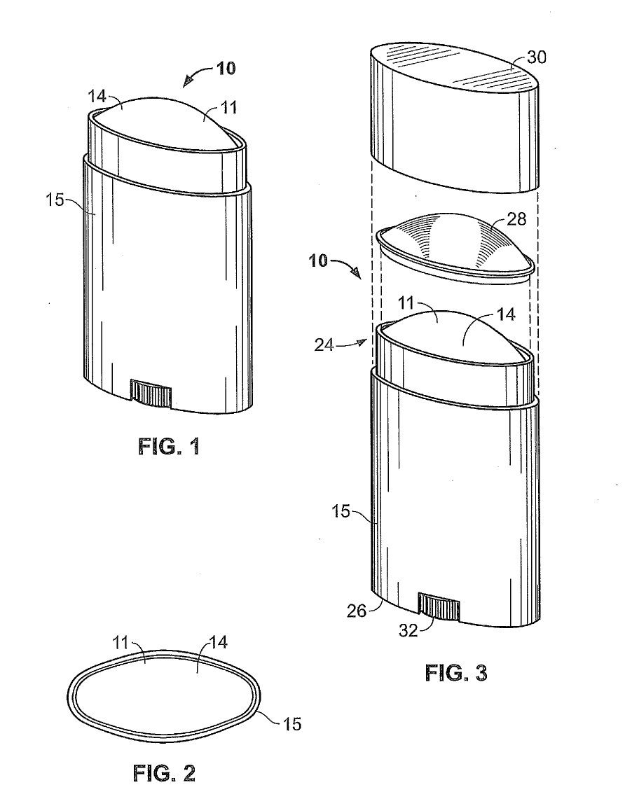

Referring to FIGS. 1 and 2, a clear stick antiperspirant product 10 in accordance with an exemplary embodiment is provided. The clear stick antiperspirant product 10 comprises a clear stick antiperspirant composition 11 in the form of a clear stick. As illustrated, the clear stick antiperspirant composition 11 has an application surface 14 that is configured to be applied to skin, such as, for example, an underarm. The clear stick antiperspirant product 10 may also comprise a container or dispenser 15 for dispensing the clear stick antiperspirant composition 11 to the skin.

The clear stick antiperspirant composition 11 contains a solvent carrier and a gelling agent that together provide a matrix, or body, of the clear stick. In an exemplary embodiment, the solvent carrier comprises a polyhydric alcohol that has from 3 to 6 carbon atoms and from 2 to 6 hydroxyl groups, such as 1,2-propylene glycol (also referred to simply as propylene glycol), dipropylene glycol, 1,3-butylene glycol, and/or the like. In one example, the solvent carrier is propylene glycol. In an exemplary embodiment, the clear stick antiperspirant composition 11 comprises the solvent carrier present in an amount of from about 50 to about 98 wt. % of the clear stick antiperspirant composition 11.

In an exemplary embodiment, dibenzylidene sorbitol (DBS) is the gelling agent. Dibenzylidene sorbitol is an effective nucleator and is typically in the form of a fine white powder. Dibenzylidene sorbitol is commercially available from several manufacturers including under the product name Geniset® D from Rika International Ltd., headquartered in Japan. Advantageously, in an exemplary embodiment, the clear stick antiperspirant composition 11 comprises dibenzylidene sorbitol at a concentration level effective to produce a clear stick with exceptional clarity and adequate hardness and/or body. For example, the clear stick antiperspirant composition 11 comprises the gelling agent present in an amount of from about 0.5 to about 3 wt. % of the clear stick antiperspirant composition 11.

The clear stick antiperspirant composition 11 may also include an additional gelling agent (e.g., one or more co-gelling agent or co-gellant) to provide additional stick hardness even when the clear stick antiperspirant composition 11 includes relatively low levels of dibenzylidene sorbitol. In one embodiment, the combined use of the co-gellant with reduced amounts of dibenzylidene sorbitol (e.g., amounts of about 1.5 wt. % or less) enable the production of clear sticks with exceptional clarity and stability. In an exemplary embodiment, the co-gellant comprises hydroxyalkyl cellulose that includes alkyl groups with 1 to 5 carbon atoms, such as hydroxypropyl cellulose. Hydroxypropyl cellulose is commercially available under the product name Klucel® HFF from Ashland Inc., headquartered in Covington, Ky. In an exemplary embodiment, the clear stick antiperspirant composition 11 comprises the co-gellant present in an amount of from about 0.08 to about 1 wt. % of the clear stick antiperspirant composition 11.

The clear stick antiperspirant composition 11 contains at least one active ingredient (i.e., active antiperspirant compound), typically metal salts, that are thought to reduce perspiration by diffusing through the sweat ducts of eccrine glands and apocrine glands and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, such as inorganic and organic salts of aluminum, zirconium, and zinc, as well as mixtures thereof Some examples are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof Exemplary aluminum salts include those having the general formula Al₂(OH)ₐCl_{b}x (H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Some zirconium salt examples are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In an exemplary embodiment, the active antiperspirant compound comprises aluminum chlorohydrate and/or aluminum-zirconium chlorohydrate. As used herein, the term “aluminum-zirconium chlorohydrate” is understood to include the tri-, tetra-, penta- and octa-chlorohydrate forms.

In an exemplary embodiment, the active antiperspirant compound is in a perspiration-reducing effective amount. For example, the clear stick antiperspirant composition 11 comprises the active antiperspirant compound present in the amount of from about 1 to about 25 wt. % of the clear stick antiperspirant composition 11.

The clear stick antiperspirant composition 11 contemplated herein also may include other additives, such as, for example, one or more emollient(s), chelating agent(s), fragrance(s), and/or the like. The emollients help provide the clear stick antiperspirant composition 11 with desirable application properties such as smoothness, reduced tack, and the like. Examples of emollients include fatty acid esters such as isopropyl myristate and isopropyl palmitate; diesters of adipic, phthalic, and sebacic acids such as di-n-butyl phthalate, diisopropyl sebacate, diethyl sebacate, and diisopropyl adipate; propylene glycol diesters of short chain fatty acids; nonvolatile silicone oils such as dimethyl siloxane and dimethicone copolyol; volatile silicones such as Dow Corning 344 and Dow Corning 345 (available from Dow Corning), Silicone 7207 and Silicate 7158 (available from Union Carbide), and SF 1202 (available from General Electric); C₁₂,-C₁₅ alcohol benzoates such as Finsolv (available from Finetex, Inc.); fatty alcohols such as cetyl alcohol and stearyl alcohol; alkyl ether derivatives of polyethylene glycols, polypropylene glycols and polypropylene polyethylene glycol copolymers such as PPG-5-Buteth-7, PPG-5-Ceteth-20, PPG-3-1sosteareth-9 and Glycereth-7-Diisononanoate. Many other examples of emollients are known in the art. In an exemplary embodiment, the clear stick antiperspirant composition 11 includes a sufficient quantity of emollient to provide a clear stick with the desired application properties without interfering with the clarity of the product. In an exemplary embodiment, the clear stick antiperspirant composition 11 comprises an emollient present in an amount of from about 0.25 to about 3 wt. % of the clear stick antiperspirant composition 11.

The chelating agents reduce the color, e.g., yellow color, of the clear stick antiperspirant composition 11 that can result, for example, from the presence of residual iron (or other metal contaminants) that may be present from a variety of sources. Examples of chelating agents include EDTA (ethylenediaminetetraacetate) salts such as Na₄. EDTA and Na₃ EDTA; hydroxyethylethylenediaminetriacetate (HEDTA); diethylenetriaminepentaacetate (DTPA); nitrilotriacetate (NTA); ethanoldiglycine disodium salt (EDG); diethanoiglycine sodium salt (DEG); and 1,3-propylenediaminetetraacetic acid (PDTA). In an exemplary embodiment, the clear stick antiperspirant composition 11 comprises a chelating agent present in an amount of from about 0.05 to about 2 wt. % of the clear stick antiperspirant composition 11.

The fragrances can be any conventional fragrance that provides the clear stick antiperspirant composition 11 with a desired scent. The quantity of fragrance included should be the quantity needed to provide the desired scent. In an exemplary embodiment, the clear stick antiperspirant composition 11 comprises a fragrance present in an amount of about 0.25 to about 2.5 wt. % of the clear stick antiperspirant composition 11.

The above list of materials is by way of example only and is not intended to be a comprehensive list of all potential components of the antiperspirant products contemplated herein. Other antiperspirant and/or deodorant components are readily identifiable to those skilled in the art and may be used in accordance with the various embodiments contemplated herein.

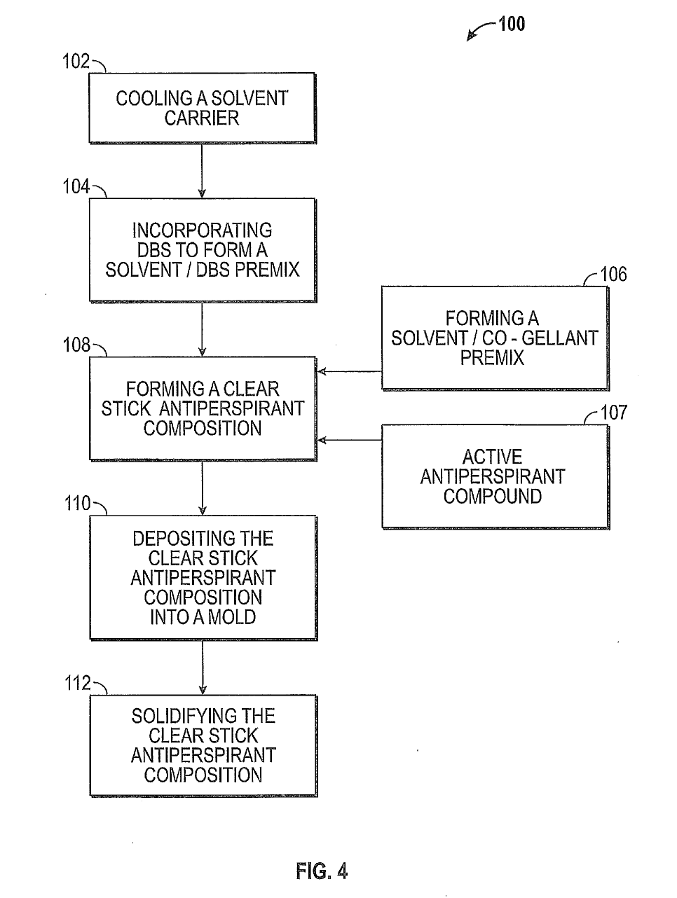

In accordance with an exemplary embodiment, a method 100 for making the clear stick antiperspirant product 10 illustrated in FIGS. 1-2 is shown in FIGS. 3-4. With reference to FIG. 4, the method 100 comprises providing a solvent carrier (step 102) to define a cool solvent carrier. In one example, the solvent carrier is propylene glycol and a quantity of propylene glycol is cooled to substantially below room temperature in a mixing container while being continuously agitated or mixed. In an exemplary embodiment, the solvent carrier is cooled to a temperature of about 12° C. or less, such as from about 0 to about 12° C., for example from about 0 to about 4° C.

A gelling agent comprising dibenzylidene sorbitol is incorporated into the cool solvent carrier to form a solvent/DBS premix (step 104). In one example, the cool solvent carrier is continuously mixed while dibenzylidene sorbitol is gradually added to the cool solvent carrier. In an exemplary embodiment, the cool solvent carrier and the dibenzylidene sorbitol are mixed together (e.g., using relatively low shear mixing) for a time of from about 5 to about 10 minutes while the temperature is maintained at about 15° C. or less, such as about 12° C. or less, such as from about 0 to about 12° C., such as from about 0 to about 10° C., such as from about 0 to about 4° C., for example about 4° C., to fully disperse and/or suspend the dibenzylidene sorbitol into the solvent carrier and form the solvent/DBS premix. In an exemplary embodiment, the solvent/DBS premix comprises the solvent carrier present in an amount of from about 70 to about 90 wt. %, such as about 75 to about 85 wt. %, and dibenzylidene sorbitol present in an amount of from about 10 to about 30 wt. %, such as about 15 to about 25 wt. % of the solvent/DBS premix.

As illustrated, in an embodiment, a solvent/co-gellant premix is formed (step 106). In one example, a quantity of solvent carrier, e.g., propylene glycol, is added to a mixing container and is continuously agitated or mixed while a co-gelling agent, such as hydroxypropyl cellulose, is gradually added to the solvent carrier. With continuous mixing, the solvent carrier and the co-gelling agent are heated to a temperature of from about 90 to about 100° C., such as from about 90 to about 95° C., to solubilize the co-gelling agent in the solvent carrier and form the solvent/co-gellant premix. In an exemplary embodiment, the solvent/co-gellant premix comprises the solvent carrier present in an amount of from about 98 to about 99.75 wt. % and the co-gelling agent present in an amount of from about 0.25 to about 2 wt. % of the solvent/co-gellant premix.

An active antiperspirant compound, e.g., aluminum chlorohydrate, aluminum-zirconium chlorohydrate, and/or the like, is provided (step 107). A clear stick antiperspirant composition is formed (step 108) by combining the active antiperspirant compound and the solvent/DBS premix to form a solvent/gellant-antiperspirant mixture. In an exemplary embodiment, the solvent/co-gellant premix and/or additional antiperspirant ingredients as discussed above in the foregoing paragraphs are combined with the active antiperspirant compound and the solvent/DBS premix at a temperature of from about 18 to about 35° C. to form the solvent/gelling-antiperspirant mixture. In one example, a quantity of the solvent carrier, e.g., propylene glycol, at about room temperature is added to a mixing container and is continuously agitated or mixed. A chelating agent, e.g., Na₄ EDTA, is added to the solvent carrier and mixed for about 5 minutes until the solution is uniform. Emollient(s), e.g., diisopropyl sebacate and/or PEG/PPG-14/4 dimethicone, and the solvent/DBS premix are then added and mixed until the solution is uniform. The solvent/co-gellant premix is then gradually added and mixed until the solution is uniform to form the solvent/gellant-antiperspirant mixture. In an exemplary embodiment, the solvent/gellant-antiperspirant mixture is then heated to a temperature of from about 90 to about 95° C., for example about 92° C., and the temperature is maintained for about 8 to about 12 minutes to substantially solubilize the ingredients including the dibenzylidene sorbitol into the solvent carrier, forming the clear stick antiperspirant composition in a molten form. In an exemplary embodiment, the clear stick antiperspirant composition comprises the solvent/DBS premix present in an amount of from about 2 to about 12 wt. %, such as about 4 to about 8 wt. %, and the solvent/co-gellant premix present in an amount of from about 20 to about 60 wt. % of the clear stick antiperspirant composition. In an exemplary embodiment, the clear stick antiperspirant composition is partially cooled to a temperature of about 70 to about 80° C. (e.g., in which the partially cooled clear stick antiperspirant composition is in a molten form) and a fragrance and/or odor neutralizer is added to the clear stick antiperspirant composition.

Referring to FIGS. 3-4, the clear stick antiperspirant composition 11 is deposited in the molten form into a mold (step 110) and allowed to solidified (step 112). The pour temperature (e.g., molten/liquidus temperature) of the clear stick antiperspirant composition 11 is generally in the range about 50° C. or greater. The clear stick antiperspirant composition 11 is cooled below about 50° C., to produce the clear stick antiperspirant product 10 in a solid form. The container 15 may be used as the mold for the clear stick antiperspirant product 10 illustrated in FIG. 3. It will be appreciated, however, that the invention is not limited to use of the container 15 as a mold and that any satisfactory mold may be used for manufacturing the clear stick antiperspirant product.

In an exemplary embodiment, the container 15 has an application end 24 and an opposite end 26. The container 15 also contains a factory seal 28, which is positioned over the application surface 14 of antiperspirant composition 11 to protect it during shipment and to render it tamper-proof prior to purchase, and a cover 30. The factory seal 28 also keeps the clear stick antiperspirant composition 11 when in a molten form within the container 15 walls and helps mold the clear stick antiperspirant composition 11 into an aesthetically pleasing application surface 14 for application to skin such as under arms. The factory seal 28 is removed by the user, and the cover is used during storage of the product between uses. As the product is exhausted, it is advanced from the container 15 by the user using an advancement device 32, e.g., a screw mechanism as shown, at the opposite end 26 of container 15.

In an exemplary embodiment, the clear stick antiperspirant composition 11 is molded into the container 15 by first sealing the application end 24 of the container 15 with the factory seal 28 and introducing a predetermined quantity of molten/liquidus clear stick antiperspirant composition 11 through the open opposite end 26. The clear stick antiperspirant composition 11 is then cooled to its solid form for example, by passing the filled container through a forced air tunnel operating at between about 10 to about 25° C. The finished product (FIG. 3) is completed by sealing the open opposite end 26 with a package base (not shown) that includes the advancement device 32. Other suitable methods for molding and/or forming the clear stick antiperspirant product known to those skilled in the art may also be used.

The following are examples of clear stick antiperspirant products in accordance with various exemplary embodiments. The examples are provided for illustration purposes only and are not meant to limit the various embodiments of the clear stick antiperspirant products in any way. All materials are set forth in weight percent.

### EXAMPLES

### Clear Stick Antiperspirant Compositions

Clear Stick Antiperspirant Product—Clear Stick Formulation (Theoretical Yield)

| Ingredient | Wt. % |
|---|---|
| Propylene Glycol | 50 to 98 |
| Aluminum Zirconium | 1 to 25 |
| Tetrachlorohydrex | |
| Odor Neutralizer | 0.25 to 3 |
| Hydroxypropyl Cellulose | 0.08 to 1 |
| Tetrasodium EDTA | 0.05 to 2.5 |
| Dibenzylidene Sorbitol | 0.5 to 3 |
| Diisopropyl Sebacate | 0.25 to 3 |
| Fragrance | 0.25 to 3 |
| PEG/PPG-14/4 Dimethicone | 0.05 to 0.75 |
| Total | 100.0 |

Clear Stick Antiperspirant Product—Clear Stick Formulation (Showing Premixes)

| Ingredient | Wt. % |
|---|---|
| Propylene Glycol | 5 to 40 |
| AAZG 3111* | 20 to 70 |
| Diisopropyl Sebacate | 0.25 to 3 |
| PEG/PPG-14/4 Dimethicone | 0.05 to 0.75 |
| Fragrance | 0.25 to 3 |
| Odor Neutralizer | 0.25 to 3 |
| Tetrasodium EDTA | 0.05 to 2.5 |
| Geniset ® D Premix** | 2 to 12 |
| Klucel ® Premix*** | 20 to 60 |
| Total | 100.0 |
| Propylene Glycol | 70 to 90 |
| Dibenzylidene Sorbitol (Geniset ® D) | 10 to 30 |
| Total | 100.0 |
| Propylene Glycol | 98 to 99.75 |
| Hydroxypropyl Cellulose (Klucel ®) | 0.25 to 2 |
| Total | 100.0 |

*AAZG 3111—is an active antiperspirant premix/mixture comprising from about 19.8 to about 24.2 wt. % aluminum zirconium tetrachlorohydrex and from about 75.8 to about 80.2 wt. % propylene glycol.
**Solvent/DBS Premix—(also referred to as “Geniset ® D Premix”)
***Solvent/Co-Gellant Premix—(also referred to as “Klucel ® Premix”)

Accordingly, methods for making clear stick antiperspirant compositions and products have been described. In an exemplary embodiment, a clear stick antiperspirant composition is formed by providing a solvent carrier that is substantially below room temperature to define a cool solvent carrier. A gelling agent comprising dibenzylidene sorbitol is incorporated into the cool solvent carrier to form a solvent/DBS premix. An active antiperspirant compound and optionally other antiperspirant ingredients are combined with the solvent/DBS premix to form a solvent/gellant-antiperspirant mixture. In an exemplary embodiment, the solvent/gellant-antiperspirant mixture is heated to substantially solubilize the ingredients into the solvent carrier to form the clear stick antiperspirant composition in a molten form. The clear stick antiperspirant composition may be deposited into a container and allowed to solidify to form a clear stick antiperspirant product.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

What is claimed is:

1. A method for making a clear stick antiperspirant composition, the method comprising the steps of:
providing a solvent carrier at substantially below room temperature to define a cool solvent carrier;
incorporating a gelling agent comprising dibenzylidene sorbitol into the cool solvent carrier to form a solvent/DBS premix; and
combining an active antiperspirant compound and the solvent/DBS premix.

2. The method of claim 1, wherein the step of providing comprises providing the solvent carrier having a temperature of about 15° C. or less.

3. The method of claim 1, wherein the step of providing comprises providing the solvent carrier having a temperature of from about 0 to about 12° C.

4. The method of claim 1, wherein the step of providing comprises providing the solvent carrier that comprises a polyhydric alcohol having from 3 to 6 carbon atoms and from 2 to 6 hydroxyl groups.

5. The method of claim 1, wherein the step of combining comprises combining the active antiperspirant compound that comprises aluminum chlorohydrate(s), aluminum chlorohydrex's, aluminum-zirconium chlorohydrate(s), and/or aluminum-zirconium chlorohydrex's with the solvent/DBS premix.

6. The method of claim 1, wherein the step of combining comprises combining the active antiperspirant compound with the solvent/DBS premix to form a solvent/gellant-antiperspirant mixture, and wherein the method further comprises the step of:
heating the solvent/gellant-antiperspirant mixture to form the clear stick antiperspirant composition.

7. The method of claim 6, wherein the step of heating comprises heating the solvent/gellant-antiperspirant mixture to a temperature of from about 90 to about 95° C. to form the clear stick antiperspirant composition.

8. A method for making a clear stick antiperspirant composition, the method comprising the steps of:
mixing a cool amount of a solvent carrier and a gelling agent that comprises dibenzylidene sorbitol at a first temperature that is substantially below room temperature to form a solvent/DBS premix;
mixing an active antiperspirant compound and the solvent/DBS premix at a second temperature to form a solvent/gellant-antiperspirant mixture, wherein the second temperature is greater than the first temperature; and
heating the solvent/gellant-antiperspirant mixture to a third temperature that is greater than the second temperature to form the clear stick antiperspirant composition in molten form.

9. The method of claim 8, wherein the step of mixing the cool amount comprises maintaining the first temperature at about 12° C. or less to form the solvent/DBS premix.

10. The method of claim 8, wherein the step of mixing the cool amount comprises mixing the cool amount of the solvent carrier and the gelling agent together for a time of from about 5 to about 10 minutes.

11. The method of claim 8, wherein the step of mixing the cool amount comprises forming the solvent/DBS premix that comprises the solvent carrier present in a first amount of from about 70 to about 90 wt. % and dibenzylidene sorbitol present in a second amount of from about 10 to about 30 wt. % of the solvent/DBS premix.

12. The method of claim 8, wherein the step of mixing the active antiperspirant compound comprises maintaining the second temperature at from about 18 to about 35° C.

13. The method of claim 8, wherein the step of mixing the active antiperspirant compound comprises mixing the active antiperspirant compound, the solvent/DBS premix, and an additional amount of the solvent carrier to form the solvent/gellant-antiperspirant mixture, and wherein the step of heating comprises forming the clear stick antiperspirant composition that comprises the solvent carrier present in a first amount of from about 50 to about 98 wt. %, dibenzylidene sorbitol present in a second amount of from about 0.5 to about 3 wt. %, and the active antiperspirant compound present in a third amount of from about 1 to about 25 wt. % of the clear stick antiperspirant composition.

14. The method of claim 8, wherein the step of heating comprises heating the solvent/gellant-antiperspirant mixture to the third temperature of from about 90 to about 95° C.

15. The method of claim 8, wherein the step of heating comprises mixing the solvent/gelling-antiperspirant mixture at the third temperature for a time of about 8 to about 12 minutes.

16. The method of claim 8, further comprising the steps of:
cooling the clear stick antiperspirant composition after the step of heating to form a partially cool clear stick antiperspirant composition; and
incorporating an odor neutralizer and/or a fragrance into the partially cool clear stick antiperspirant composition.

17. The method of claim 16, wherein the step of cooling comprises forming the partially cool clear stick antiperspirant composition having a temperature of from about 70 to about 80° C.

18. The method of claim 8, further comprising the step of:
mixing and heating a co-gelling agent with an additional amount of the solvent carrier to form a solvent/co-gellant premix, and wherein the step of mixing the active antiperspirant compound comprises mixing the active antiperspirant compound, the solvent/DBS premix, and the solvent/co-gellant premix to form the solvent/gellant-antiperspirant mixture as a solvent/gellant-antiperspirant solvent/co-gellant mixture.

19. The method of claim 18, wherein the step of mixing and heating comprises heating the co-gelling agent and the additional amount of the solvent carrier to a temperature of from about 90 to about 100° C.

20. A method for making a clear stick antiperspirant product, the method comprising the steps of:
forming a clear stick antiperspirant composition comprising:
incorporating a gelling agent comprising dibenzylidene sorbitol into a cool solvent carrier that is substantially below room temperature to form a solvent/DBS premix;
combining an active antiperspirant compound and the solvent/DBS premix to form a solvent/gellant-antiperspirant mixture; and
heating the solvent/gellant-antiperspirant mixture to form the clear stick antiperspirant composition in molten faun;
depositing the clear stick antiperspirant composition into a mold; and
allowing the clear stick antiperspirant composition to solidify.
